(19) United States
(12) Patent Application Publication
(10) Pub. No.: US 2019/0300927 A1
(43) Pub. Date: Oct. 3, 2019

(54) METABOLIC ASSAY FOR BACTERIAL GROWTH AND GRAM TYPING

(71) Applicant: SELUX DIAGNOSTICS, INC., Charlestown, MA (US)

(72) Inventors: Benjamin Spears, Charlestown, MA (US); Eric Stern, Charlestown, MA (US); Kelly Flentie, Charlestown, MA (US)

(21) Appl. No.: 16/366,244

(22) Filed: Mar. 27, 2019

Related U.S. Application Data
(60) Provisional application No. 62/648,826, filed on Mar. 27, 2018.

(51) Int. Cl. C12Q 1/20; C12Q 1/08

(52) U.S. Cl. CPC C12Q 1/20; C12Q 1/08

(57) ABSTRACT

The use of metabolic probes is well-established for determining cell viability and assessing drug cytotoxicity. Resazurin-based formulations, in particular, have found utility for determining susceptibilities of microorganisms to antimicrobials, specifically through their use in antibiotic susceptibility testing (AST). There is a strong need currently to shorten AST durations, thus resazurin formulations that produce signals earlier are advantageous. This need results from the slow state of clinical microbiology testing, which may leave patients exposed to unnecessary or ineffective broad-spectrum agents for prolonged periods of time. Another slow microbiology test is Gram staining, still most often performed manually in sequential steps. Speeding Gram typing, preferably with an automated platform, would also speed time-to-results and decrease manual workloads on medical technologists in clinical microbiology laboratories.

### CROSS REFERENCE TO RELATED APPLICATIONS

This application claims priority to U.S. provisional application 62/648,826, filed on Mar. 27, 2018, and titled “Metabolic Assay for Bacterial Growth and Gram Typing.” The foregoing application is incorporated by reference herein in its entirety and for all purposes.

### FIELD OF THE DISCLOSURE

This disclosure relates to the preparation and testing of clinical microbiological samples.

### BACKGROUND

The use of resazurin-based formulations for bacterial growth determinations and AST is well-established. Resazurin, which absorbs blue light but exhibits no fluorescence, is a redox-sensitive compound that can be reduced to fluorescent resorufin (Ex=560 nm, Em=590 nm) by cellular processes. Thus, cellular viability can be monitored by increases in fluorescence intensity.

“Alamar Blue™” became a commercially-available resazurin formulation through the advances described in U.S. Pat. No. 5,501,959, which included ferricyanide and ferrocyanide salts together with methylene blue as stabilizers to prevent resazurin reduction during storage. The patent describes the ferricyanide, ferrocyanide, and methylene blue as “poising agents” that inhibit resazurin reduction and should be kept to a minimum concentration. As such, the useful concentration range (w/w) of methylene blue is defined as ⅕^{th} to 1/10^{th} that of resazurin.

Although this Alamar Blue formulation meets necessary stability criteria, Pseudomonas aeruginosa, an important non-fastidious gram-negative bacterial pathogen, does not effectively metabolize it, FIG. 1. Furthermore, multiple formulations of Alamar Blue are now available from commercial distributors, but none of those tested were capable of detecting P. aeruginosa growth in <5 hours, FIG. 1.

Another frequently used molecule for the determination of bacterial viability is iodonitrotetrazolium (INT), which is converted to a highly absorbent Forazan (Abs.=490 nm) upon reduction by an actively metabolizing bacterium. However, though this molecule is readily metabolized by other species of bacteria, it is not capable of detecting P. aeruginosa growth in <5 hours, FIG. 1.

### SUMMARY

The present disclosure provides methods for assaying microorganisms using resazurin. In one embodiment, this method may include incubating the microorganism under conditions promoting microorganism growth in a reservoir comprising nutrient broth in the presence of a metabolic probe formulation. This metabolic probe formulation may comprise resazurin at a concentration C_{R}, one or more stabilizing salts that maintain the potential of the growth media between +0.3 and +0.45 volts in the absence of cellular growth, and one or more enhancing agents that maintain the redox potential of the growth media above −0.1 volts and are present at a concentration C_{E}, where C_{E}≥0.5×C_{R}. The method may further comprise measuring the fluorescence of resorufin at one or more timepoints. The salts may be potassium ferrocyanide, ferric, and/or ferricenium. The salts may be present in both oxidized and reduced forms. The salt pair may be any of potassium ferricyanide, potassium ferrocyanide, ferrous/ferric, and ferricenium/ferrocene.

In an embodiment of this disclosure, the one or more enhancing agents that maintain the redox potential of the growth media above −0.1 volts are selected to inhibit reduction of resorufin to dihydroresorufin. The enhancing agents are redox indicators selected from the group consisting of methylene blue, toluidine blue, azure I, and gallocyanine. The resazurin concentration, C_{R}, may be selected to be sufficient to be detectable while being substantially non-toxic to cell growth. The concentration of the enhancing agents, C_{E}, may be set such that C_{E}≥C_{R}; C_{E}≥2×C_{R}; C_{E}≥5×C_{R}; C_{E}≥10×C_{R}. The conditions that promote microorganism growth may comprise a temperature in the range of 33-37° C. The fluorescence measurement of resorufin may be used to determine antimicrobial susceptibility to one or more antimicrobials.

In another aspect of this disclosure, one or more antimicrobial compounds may be present during incubation with the metabolic probe formulation. The incubation with the metabolic probe formulation may follow an incubation of the microorganism and the one or more antimicrobials in nutrient broth without the metabolic probe formulation under conditions that promote microorganism growth. Further, a 30-120-minute incubation with the metabolic probe formulation may follow a 3-9 hour incubation of the microorganism and the one or more antimicrobials in nutrient broth without the metabolic probe formulation under conditions that promote microorganism growth. The resorufin fluorescence from the metabolic probe formulation assay may be compared with data from one or more growth assays. The one or more growth assays may comprise optical density measurements, measurements of growth media pH, alternate metabolic probe measurements, ATP measurements, NADH measurements, DNA or RNA measurements, protein measurements, and/or enzymatic assays. The alternate metabolic probe may comprise resazurin at concentration C_{R2}, methylene blue at concentration C_{B2}, potassium ferricyanide and potassium ferrocyanide, such that C_{R2}≥0.5×C_{B2}. These assays may be used to indicate gram type of the microorganism. The resorufin fluorescence from the metabolic probe formulation may be measured after a 30-180-minute incubation. The microorganisms may derive from a clinical sample. The clinical sample may be selected from the list including, but not limited to, blood, cerebrospinal fluid, urine, stool, vaginal, sputum, bronchoalveolar lavage, throat, nasal/wound swabs, and combinations thereof. The microorganisms may be selected from the list including, but not limited to, Escherichia coli, Enterococcus spp., Staphylococcus spp., Klebsiella spp., Acinetobacter spp., Pseudomonas spp., Enterobacter spp., Streptococcus spp., Proteus spp., Aerococcus spp., Actinomyces spp., Bacillus spp., Bartonella spp., Bordetella spp., Brucella spp., Campylobacter spp., Chlamydia spp., Chlamydophila spp., Clostridium spp., Corynebacterium spp., Ehrlichia spp., Francisella spp., Gardenerella spp., Haemophilius spp., Helicobacter spp., Lactobacillus spp., Legionella spp., Leptospira spp., Listeria spp., Mycobacterium spp., Mycoplasma spp., Neisseria spp., Nocardia spp., Pasteurella spp., Rickettsia spp., Salmonella spp., Shigella spp., Stenotrophomonas spp., Treponema spp., Ureaplasma spp., Vibrio spp., Yersinia spp., Candida spp., Issatchenkia spp., Blastomyces spp., Coccidioides spp., Aspergillus spp., Cryptococcus spp., Histoplasma spp., Pneumocystis spp., Stachybotrys spp., Sporothrix, Exserohilum, Cladosporium, ringworm, mucormycetes, and combinations thereof. The inoculation steps may be automated. The assay steps may be automated. The resazurin may be a water-soluble salt.

In another embodiment, this disclosure describes a method for determining antimicrobial susceptibility of a microorganism comprising introducing a suspension of a microorganism to a cartridge comprising a plurality of chambers comprising one or more antimicrobials, incubating the cartridge under conditions promoting microorganism growth for an initial time period wherein the microorganism is in a reservoir comprising nutrient broth in the presence of a metabolic probe formulation, performing a checkpoint assay in at least a subset of chambers for determining whether a microorganism growth has achieved a threshold value, and upon microorganism growth achieving the threshold value, performing a surface area assay in a plurality of cartridge chambers and comparing surface area measurements between the plurality of cartridge chambers, thereby determining the susceptibility of the microorganism to a plurality of antimicrobials. The metabolic probe formulation comprises resazurin at a concentration C_{R}, one or more stabilizing salts that maintain the potential of the growth media between +0.3 and +0.45 volts in the absence of cellular growth, and one or more enhancing agents that maintain the redox potential of the growth media above −0.1 volts and are present at a concentration C_{E}, where C_{E}≥0.5×C_{R}.

In another embodiment of the disclosure, a method of assessing antimicrobial susceptibility comprises the steps of inoculating an AST panel with a patient sample, the AST panel comprising a plurality of serially diluted antimicrobials, incubating the AST panel under conditions favorable for microbial growth, performing a checkpoint assay to determine a level of microbial growth in a control well of the AST panel, if a level of microbial growth exceeds a predetermined threshold, performing a growth assay, and based on a result of the growth assay, determining the antimicrobial susceptibility of the microorganism wherein (a) the step of performing a growth assay comprises assessing a metabolic signal in each of the plurality of serially diluted antimicrobials, (b) the metabolic signal is a signal from a redox reaction, and (c) the redox reaction can be carried out by pseudomonas bacteria. In this embodiment, the step of performing a growth assay further comprises assessing the surface area of cells in each of the serially diluted antimicrobials.

### BRIEF DESCRIPTION OF THE FIGURES

FIG. 1 depicts signal to noise ratios of the tested metabolic indicators vs. the preferred embodiment formulation described here.

FIG. 2A-C depicts AST data for tetracycline with Pseudomonas aeruginosa using FIG. 2A Alamar Blue″, FIG. 2B INT, and FIG. 2C the preferred embodiment formulation.

FIG. 3 depicts the fluorescence of remaining resorufin after 4 hour incubation with 4 clinical isolates of Pseudomonas aeruginosa in MHB or MHB alone.

FIG. 4 depicts the difference of the ratio of Alamar Blue™/preferred embodiment formulation once a statistically significant amount of growth has occurred vs. the initial ratio for 10 bacterial species.

### DETAILED DESCRIPTION

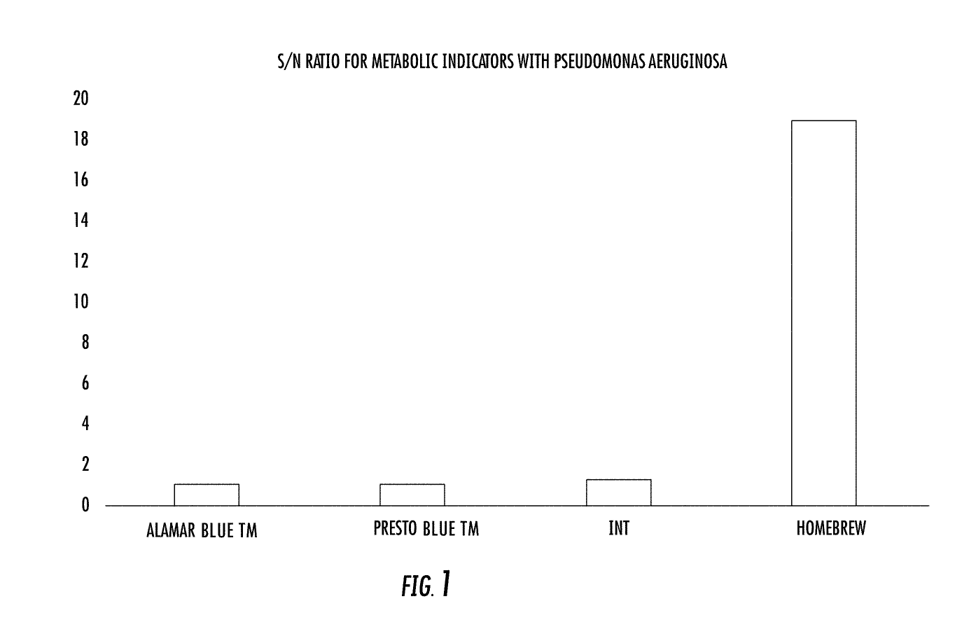

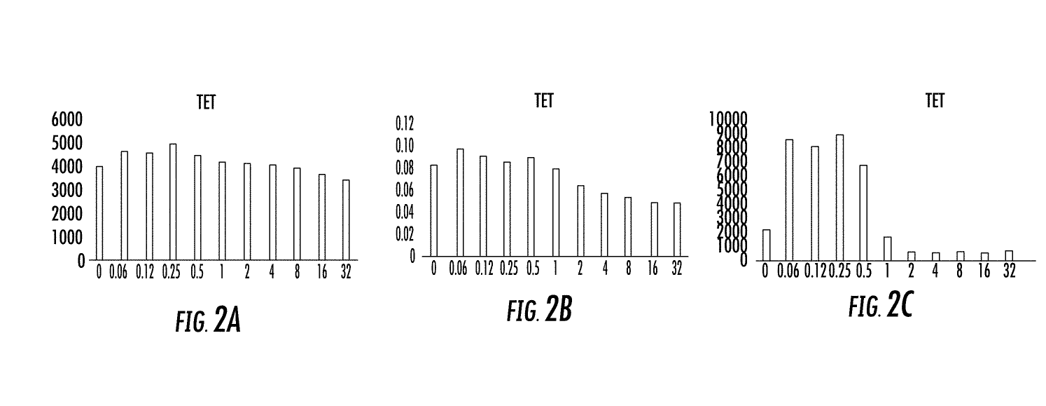

Here we introduce the surprising finding that increasing the methylene blue “poising agent” to concentrations 5-50-fold, preferably 15-fold, greater than resazurin (w/w), while keeping iron ferricyanide and iron ferrocyanide concentrations similar to commercial Alamar Blue formulations (0.0.1-0.03% w/v), enables P. aeruginosa to metabolize the resazurin, FIG. 1. The importance of this is further magnified when we compare actual AST data for the drug tetracycline that can be obtained from these different metabolic indicator solutions, FIG. 2A-C.

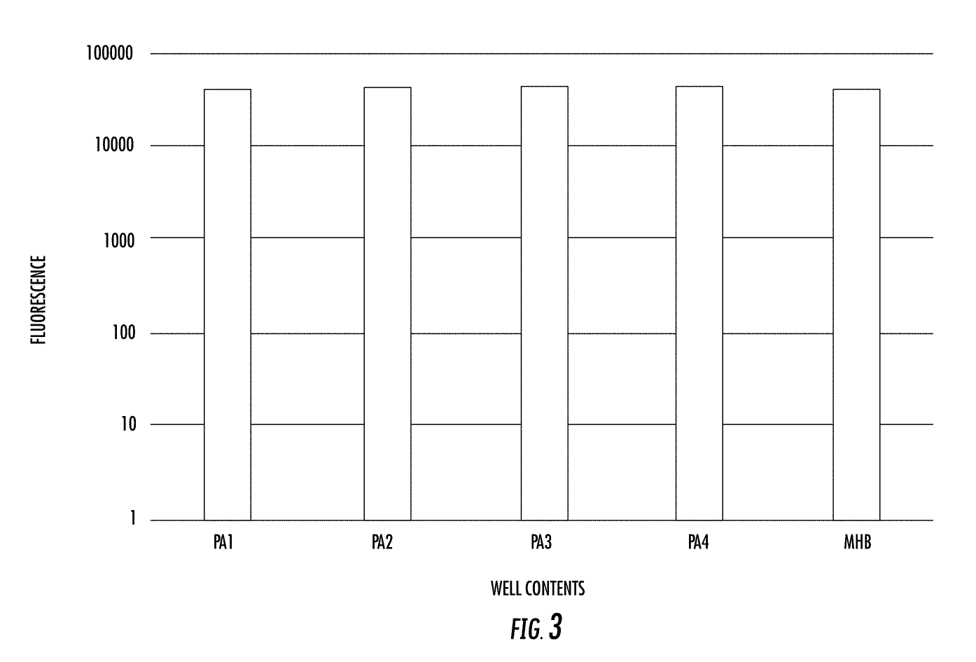

Methylene blue is included in commercial formulations to maintain the potential above −0.1 volts so as to inhibit the secondary reduction of resorufin to the non-fluorescent, uncolored dihydroresorufin. It may thus be surmised that P. aeruginosa fails to provide a strong fluorescent signal with commercial Alamar Blue formulations due to dual reductions of resazurin. To test this, resorufin alone was added to P. aeruginosa, with no methylene blue present, FIG. 3. These data demonstrate that 4-hour P. aeruginosa growth in Mueller-Hinton broth does not eliminate the resorufin fluorescence.

Thus, it is surprising that the addition of high concentrations of methylene blue to the formulation described here enables P. aeruginosa reduction of resazurin to resorufin.

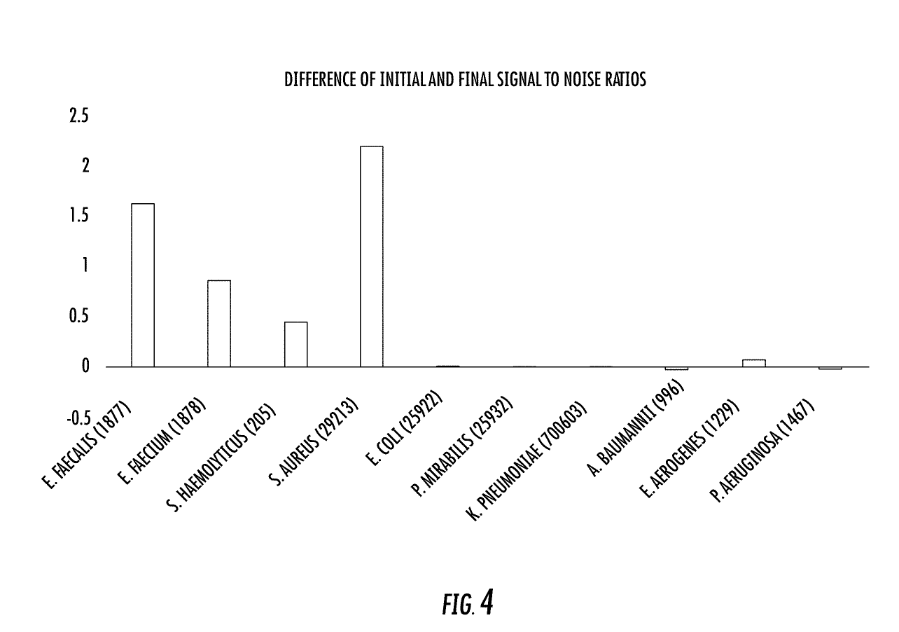

The high concentration of methylene blue in the formulation described here also enables the solution to be used for gram typing of bacteria. High concentrations of methylene blue are known to be tolerated by gram-negative organisms but inhibitory to gram-positive organisms [Fung and Miller. Appl. Microbiol. 25, 793-799 (1973)]. Studies with 10 species of non-fastidious bacteria showed that the new formulation developed here is metabolized much more rapidly by gram-negative species and negligibly by gram-positive species under the conditions of the test, FIG. 4.

The Table below shows the formulation of an embodiment for this disclosure.

| Compound | Concentration | Range |
|---|---|---|
| Resazurin | 220 uM | 10 uM to 100 mM |
| Methylene blue | 2.36 mM | 100 uM to 1M |
| Ferricyanide | 0.005% (w/v) | 0-0.1% (w/v) |
| Ferrocyanide | 0.005% (w/v) | 0-0.1% (w/v) |

Additional agents that can be used in place of methylene blue include toluidine blue, azure I, and gallocyanine. The ferricyanide to ferrocyanide molar ratios are preferably 1:1 but may range from 4:1 to 1:4. Other suitable salt pairs include ferricenium/ferrocene and ferric/ferrous salts. Alternative salts include others from the same electrochemical series that can maintain media potentials between +0.3 and +0.45 volts in the absence of cellular growth, are non-toxic and soluble at the concentrations and pH used. The resazurin is preferably an aqueous-soluble salt. The preferred embodiment is purely aqueous.

### Examples

AST Protocol: AST plates were removed from the −80° C. freezer and thawed. 0.5 McFarland dilutions were prepared. 10 μL of the bacteria dilution was added to the plate (except in well H12). 10 μL of Alamar Blue™ was added to wells G12 and H12. The plates were incubated for 3 hrs at 35° C. in the shaking incubator. After, 10 μL of Alamar Blue™, INT, or the preferred embodiment formulation was added to each well. The plate was incubated for another hour at 35° C. The plate was read at 560 nm/590 nm. Next, 150 μL of BLAST buffer was added to each well (CTAB for gram positive [or Proteus, Serratia, Morganella] or 1% PBST for gram negative). The plate was incubated at room temperature for 10 min on a shaker at 450 rpm. The plate was spun at 2,500×g for 2.5 min. The plate was aspirated and 100 μL of 1×PBST was added to each well. 10 μL of 20 ng Europium cryptate was added to each well. The plate was incubated at room temperature for 10 min on a shaker at 450 rpm. The plate was aspirated and 200 μL of 1×PBST was added to each well. The plate was spun at 2,500×g for 2.5 min. Steps 15 and 16 were repeated two more times. The plate was read at 330 nm/615 nm.

Protocol for Gram Determination: A 0.5 McFarland dilutions were prepared. The McFarlands were further diluted to 24,000 CFU/mL in 5 mL of Mueller Hinton Broth (MHB) and 150 μL was added to the plate. The first row of wells did not have any Alamar Blue™ added. Alamar Blue™ or the preferred embodiment formulation described here was added to the remaining rows. The plate was read every hour at absorbance 600 and 560 nm/590 nm. Ratios between Alamar Blue™ and the preferred embodiment formulation were calculated, and differences between the final and initial ratios were calculated.

FIG. 3 Experiment: Four clinical isolates of P aeruginosa were inoculated into MHB at a 1:200 dilution from a 0.5 McFarland standard. Resorufin was added to each well at a 100 μM final concentration. Plates were incubated shaking for 4 hours at 37° C. Fluorescence was read at Ex560/Em590. Uninoculated MIHB with resorufin was included as a negative control.

1. A method for assaying microorganisms comprising the steps of:
a. incubating the microorganism under conditions promoting microorganism growth in a reservoir comprising nutrient broth in the presence of a metabolic probe formulation comprising:
i. resazurin at a concentration C_{R},
ii. one or more stabilizing salts that maintain the potential of the growth media between +0.3 and +0.45 volts in the absence of cellular growth, and
iii. one or more enhancing agents that maintain the redox potential of the growth media above −0.1 volts and are present at a concentration C_{E}, where C_{E}≥0.5×C_{R}, and
b. measuring the fluorescence of resorufin at one or more timepoints.

2. The method of claim 1, wherein the one or more salts are selected from the group consisting of potassium ferrocyanide, ferric, and ferricenium.

3. The method of claim 1, wherein the one or more salts comprise a pair present in both oxidized and reduced forms.

4. The method of claim 3, wherein the salt pair is selected from the group consisting of potassium ferricyanide, potassium ferrocyanide, ferrous/ferric, and ferricenium/ferrocene.

5. The method of claim 1, wherein the one or more enhancing agents that maintain the redox potential of the growth media above −0.1 volts are selected to inhibit reduction of resorufin to dihydroresorufin.

6. The method of claim 1, wherein the enhancing agents are redox indicator selected from the group consisting of methylene blue, toluidine blue, azure I, and gallocyanine.

7. The method of claim 1, wherein the resazurin concentration, C_{R}, is selected to be sufficient to be detectable while being substantially non-toxic to cell growth.

8. The method of claim 1, wherein the concentration of the enhancing agents, C_{E}, is set such that C_{E}≥C_{R}; C_{E}≥2×C_{R}; C_{E}≥5×C_{R}; C_{E}≥10×C_{R}.

9. The method of claim 1, wherein the conditions that promote microorganism growth comprise a temperature in the range of 33-37° C.

10. The method of claim 1, wherein the fluorescence measurement of resorufin is used to determine antimicrobial susceptibility to one or more antimicrobials.

11. The method of claim 10, wherein the one or more antimicrobial compounds are present during incubation with the metabolic probe formulation.

12. The method of claim 10, wherein the incubation with the metabolic probe formulation follows an incubation of the microorganism and the one or more antimicrobials in nutrient broth without the metabolic probe formulation under conditions that promote microorganism growth.

13. The method of claim 12, wherein a 30-120-minute incubation with the metabolic probe formulation follows a 3-9 hour incubation of the microorganism and the one or more antimicrobials in nutrient broth without the metabolic probe formulation under conditions that promote microorganism growth.

14. The method of claim 1, wherein the resorufin fluorescence from the metabolic probe formulation assay is compared with data from one or more growth assays.

15. The method of claim 14, wherein the one or more growth assays comprise optical density measurements, measurements of growth media pH, alternate metabolic probe measurements, ATP measurements, NADH measurements, DNA or RNA measurements, protein measurements, enzymatic assays.

16. The method of claim 15, wherein the alternate metabolic probe comprises resazurin at concentration C_{R2}, methylene blue at concentration C_{B2}, potassium ferricyanide and potassium ferrocyanide, such that C_{R2}≥0.5×C_{B2}.

17. The method of claim 16, wherein the assays are used to indicate gram type of the microorganism.

18. The method of claim 1, wherein the resorufin fluorescence from the metabolic probe formulation is measured after a 30-180-minute incubation.

19. The method of claim 1 in which the microorganisms derive from a clinical sample.

20. The method of claim 1 in which the clinical sample is selected from the list including, but not limited to, blood, cerebrospinal fluid, urine, stool, vaginal, sputum, bronchoalveolar lavage, throat, nasal/wound swabs, and combinations thereof.

21. The method of claim 1 in which the microorganisms are selected from the list including, but not limited to, Escherichia coli, Enterococcus spp., Staphylococcus spp., Klebsiella spp., Acinetobacter spp., Pseudomonas spp., Enterobacter spp., Streptococcus spp., Proteus spp., Aerococcus spp., Actinomyces spp., Bacillus spp., Bartonella spp., Bordetella spp., Brucella spp., Campylobacter spp., Chlamydia spp., Chlamydophila spp., Clostridium spp., Corynebacterium spp., Ehrlichia spp., Francisella spp., Gardenerella spp., Haemophilius spp., Helicobacter spp., Lactobacillus spp., Legionella spp., Leptospira spp., Listeria spp., Mycobacterium spp., Mycoplasma spp., Neisseria spp., Nocardia spp., Pasteurella spp., Rickettsia spp., Salmonella spp., Shigella spp., Stenotrophomonas spp., Treponema spp., Ureaplasma spp., Vibrio spp., Yersinia spp., Candida spp., Issatchenkia spp., Blastomyces spp., Coccidioides spp., Aspergillus spp., Cryptococcus spp., Histoplasma spp., Pneumocystis spp., Stachybotrys spp., Sporothrix, Exserohilum, Cladosporium, ringworm, mucormycetes, and combinations thereof.

22. The method of claim 1, wherein the inoculation steps are automated.

23. The method of claim 1, wherein the assay steps are automated.

24. The method of claim 1, wherein the resazurin is a water-soluble salt.

25. A method for determining antimicrobial susceptibility of a microorganism comprising
a. introducing a suspension of a microorganism to a cartridge comprising a plurality of chambers comprising one or more antimicrobials;
b. incubating the cartridge under conditions promoting microorganism growth for an initial time period wherein the microorganism is in a reservoir comprising nutrient broth in the presence of a metabolic probe formulation comprising:
i. resazurin at a concentration C_{R},
ii. one or more stabilizing salts that maintain the potential of the growth media between +0.3 and +0.45 volts in the absence of cellular growth, and
iii. one or more enhancing agents that maintain the redox potential of the growth media above −0.1 volts and are present at a concentration C_{E}, where C_{E}≥0.5×C_{R};
c. performing a checkpoint assay in at least a subset of chambers for determining whether a microorganism growth has achieved a threshold value; and
d. upon microorganism growth achieving the threshold value, performing a surface area assay in a plurality of cartridge chambers and comparing surface area measurements between the plurality of cartridge chambers, thereby determining the susceptibility of the microorganism to a plurality of antimicrobials.

26. A method of assessing antimicrobial susceptibility comprising the steps of:
inoculating an AST panel with a patient sample, the AST panel comprising a plurality of serially diluted antimicrobials;
incubating the AST panel under conditions favorable for microbial growth;
performing a checkpoint assay to determine a level of microbial growth in a control well of the AST panel;
if a level of microbial growth exceeds a predetermined threshold, performing a growth assay; and
based on a result of the growth assay, determining the antimicrobial susceptibility of the microorganism
wherein (a) the step of performing a growth assay comprises assessing a metabolic signal in each of the plurality of serially diluted antimicrobials, (b) the metabolic signal is a signal from a redox reaction, and (c) the redox reaction can be carried out by pseudomonas bacteria.

27. The method of claim 26, wherein the step of performing a growth assay further comprises assessing the surface area of cells in each of the serially diluted antimicrobials.
